(19) United States
(12) Patent Application Publication
(10) Pub. No.: US 2007/0141006 A1
(43) Pub. Date: Jun. 21, 2007

(54) Cosmetic compositions containing modified polyamines and the uses thereof

(76) Inventors: Aude Livoreil, Paris (FR); Gabin Vic, Venette (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/593,064

(22) PCT Filed: Mar. 16, 2005
(86) PCT No.: PCT/FR05/50169

§ 371 Date: Sep. 15, 2006

(30) Foreign Application Priority Data
Mar. 17, 2004 (FR) 0450532

(51) Int. Cl. A61K 8/64; A61K 8/81; A61K 8/73

(52) U.S. Cl. 424/70.7; 424/70.14; 424/70.15; 424/70.17; 977/754

(57) ABSTRACT

The invention relates to an aqueous cosmetic composition containing, in a cosmetically acceptable medium, at least one type of polymer compound whose chain comprises at least two types of amine units —NH— and/or 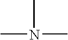 and is devoid of any vinyl amine or vinyl amide unit, wherein said polymer compound is modified with one or more hydrophilic and/or hydrophobic hydrocarbon segments, said segment(s) being different from sugar and being devoid of any sulfur, silicone or amidino group, wherein the modification with hydrophobic hydrocarbon segments is not carried out by means of a bifunctional spacer group.

This invention relates to an aqueous cosmetic composition containing one or more particular modified polyamines, as well as the use of particular polyamines for providing softness to the hair and for improving the deposition homogeneity of a cosmetic active agent onto the keratinic materials and especially onto the hair.

The use of conventional polyamines such as for example polyethyleneimines, polylysines and polyornithins, is known in the cosmetic field. These polyamines generally have a good affinity for the hair because of their cationic nature.

However, the cosmetic properties of such polyamines often are not sufficient. They suffer in particular from stickiness upon application onto the keratinic fibers, and their deposition onto the fiber is often uneven. This creates heavy feels upon application and during the shampoo process.

Moreover some modified polyamines are known, that is to say some carrying hydrophilic or hydrophobic hydrocarbon groups.

Such modified polyamines may amongst others be used for detergency purposes or so as to provide fabrics with conditioning benefits.

Thus, the international patent application WO 02/095122 discloses the use of polyethyleneimine and of polyvinyl amines that have been modified with hydrophobic groups to prevent fabrics from creasing.

The European patent application EP 0 112 593 and the U.S. Pat. No. 4,891,160 describe the use of ethoxylated polyamines in detergent compositions.

Some modified polyamines are also used in cosmetology to improve the cosmetic quality of the hair.

So, the international patent application WO 02/15854 describes the use of hydrophilic graft copolymers based on vinyl amine or vinyl amide in cosmetic compositions to provide the hair with a pleasant feel and a positive conditioning benefit.

The U.S. Pat. No. 5,756,080 describes the use of silicone polyethyleneimines to provide the hair with improved conditioning properties, such as softness for example.

The European patent application EP 0 524 612 describes the use of a polydimethylsiloxane modified with a poly(N-acylalkyleneimine) segment, to provide the hair with a shiny appearance, flexibility and softness.

The patent application JP H8-217656 describes the use of glycopolyaminoacids, especially a polyamino acid to which sugars and/or derivatives thereof are linked through a covalent bond for hair stimulation.

It is an object of the present invention to provide new compositions containing polyamines modified with one or more hydrocarbon hydrophilic and/or hydrophobic segments, such modified polyamines resulting in an improvement of the cosmetic quality of the hair, especially by providing softness, shine, without any sticky feel, and distributing more evenly onto the keratinic materials and especially the hair, and making it also possible to improve the deposition homogeneity of a cosmetic active agent onto the keratinic materials.

It is thus an object of the present invention to first provide an aqueous cosmetic composition containing, in a cosmetically acceptable medium, at least one type of polymer compound whose chain comprises at least two types of amine units —NH— and/or 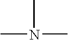
and is devoid of any vinyl amine or vinyl amide unit, said polymer compound being modified with one or more hydrophilic and/or hydrophobic hydrocarbon segments, said segment(s) being different from sugar and being devoid of any sulfur, silicone or amidino group, wherein the modification with a hydrophobic hydrocarbon segment is not carried out by means of a bifunctional spacer group.

As used herein, “a polymer compound modified with one or more hydrophilic and/or hydrophobic hydrocarbon segments” means a polymer compound that is chemically bound to one or more hydrophilic and/or hydrophobic hydrocarbon segments.

As used herein, “a sugar segment” means any mono-, oligo- or polysaccharide segment.

As used herein, “an amidino group” means a group having following formula:
—C(═NH)—NH₂

The use in cosmetology of these modified polymer compounds enables to combine the great affinity of the amine parts for the hair with the cosmetic properties provided for by the hydrophilic or hydrophobic segments, such as softness, shine, and the lack of stickiness.

The polymer compounds that can be used in the composition of the invention may be linear, branched, hyper-branched or dendrimeric.

Hyper-branched polymers are molecular constructions having a branched structure, in general around a core. Their structure in general does not have any symmetry: the basic units, or monomers, that have been used to construct the hyper-branched polymer may be different in nature and their distribution be not regular. The nature and the length of the polymer branches may differ. The number of basic units, or monomers, may differ depending on the different branchings. While being asymmetric, hyper-branched polymers may have a highly branched structure around a core, successive branching layers or generations; a terminal chain layer.

Hyper-branched polymers do generally result from the polycondensation of one or more ABx monomers, A and B being reactive groups that may react with one another, x being an integer equal or higher than 2, but other preparation methods may also be envisaged. Hyper-branched polymers are characterized by their polymerization degree PD=1-b, b representing the percentage of the B non-terminal functionalities that do not have react with an A group. Condensation being non systematic, as opposed to the dendrimer synthesis, the polymerization degree is less than 100%. As conventionally determined by known synthesis methods the polymerization degree does usually range from 15 to 90%.

A T-terminal group may also be reacted in the hyper-branched polymer so as to obtain a particular functionality at the end of the chains.

Many hyper-branched polymers may be associated with one another by means of a covalent bond or using a different type of bond, through their end groups. Such so called bridged polymers do belong to the definition of the hyper-branched polymers according to the present invention.

Dendrimers are highly branched polymers and oligomers having a well defined chemical structure that are said to be “perfect” hyper-branched polymers.

In general dendrimers comprise a core, a determined number of branching generations or spindles and end groups. Spindle generations are made of structural units that are the same for one given spindle generation and that may be the same or different for different spindle generations. Spindle generations do radially extend according to a geometric progression from the core. The end groups of a n-generation dendrimer are functional end groups of the n-generation, or end generation, spindles.

The dendrimer definition as hereabove defined does include symmetrical branching molecules. Also included are non symmetrical branching molecules, as for example dendrimers which spindles are lysine groups wherein the branching of a spindle generation on the preceding one occurs on the lysine α- and ε-amines, which results in a spindle length difference for the different branchings.

The so called “dense star polymers”, “starburst polymers”, and “rod-shaped dendrimers”, are included in the definition of dendrimers as used herein. The so called arborol molecules and cascade molecules also belong to the definition of dendrimers as used herein.

Several dendrimers may be connected to each other by means of a covalent bond or using a different type of bond, through their end groups so as to obtain entities that are known as being “bridged dendrimers” or “dendrimer clusters”. Such entities are included in the definition of dendrimers as used herein.

Dendrimers may come as a group of molecules belonging to the same generation and then are said to be monodispersed; they also may come as groups belonging to different generations and then are said to be polydispersed. The definition of dendrimers as used herein does include both monodispersed and polydispersed groups of dendrimers.

As explained hereabove, the composition of the invention comprises at least one polymer compound modified with one or more hydrophilic and/or hydrophobic segments.

In the following description, the polymer compounds that may be used are first described, then the hydrophilic and hydrophobic segments with which such polymer compounds may be modified.

The polymer compounds contained in the composition of the invention may be selected from:

polyalkyleneimines, preferably poly((C₂-C₅)alkylene imines),

polymers that are grafted by a (C₂-C₅)alkylene imine, preferably polymers that are grafted by an ethyleneimine, more preferably polyamidoamines, crosslinked or not, grafted by the ethyleneimine,

amino (C₁-C₄)alkyl(meth)acrylate based copolymers, preferably of aminoethyl(meth)acrylate type,

polyallyl amines,

polycondensates from at least one compound selected from piperazine, 1-(2-aminoethyl)piperazine, 1,4-bis(3-aminopropyl)piperazine), (C₁-C₂₅)1-alkylpiperazine, 1,4-di((C₁-C₂₅)alkyl)piperazine, 1-(2-hydroxy((C₂-C₂₅)alkyl))piperazine, imidazole, C₁-C₂₅ alkyl imidazole, or combinations thereof, with at least one compound selected from a C₆-C₂₂ alkylene dihalide, an epihalohydrine and/or a C₈-C₂₂ bisepoxide,

polymers containing at least two units of one or more basic amino acids, and

primary amine-containing dendrimers.

Polyalkyleneimines that can be used in the composition of the invention include polyethyleneimines and polypropyleneimines.

Polyethyleneimines (PEI) that can be used in the invention have generally the following formula:
—(CH₂—CH₂—NH)ₙ—
wherein n corresponds to the ethyleneimine unit average number, n ranging from 5 to 10 000.

Ethyleneimine homopolymers may be branched.

PEI-7 (n=7), PEI-15 (n=15), PEI-30 (n=30), PEI-45 (n=45), PEI-275 (n=275), PEI-700 (n=700), PEI-1000 (n=1000), PEI-1400 (n=1400), PEI-1500 (n=1500), PEI-1750 (n=1750), and PEI-2500 (n=2500) especially have to be mentioned.

A description of the polyethyleneimines can be found inter alia in KIRK-OTHMER ENCYCLOPEDIA OF CHEMICAL TECHNOLOGY, third Edition, vol. 20, 1982, p. 214-216, and in Polyethyleneimine Prospective Application, H. N. Feigenbaum, Cosmetics & Toiletries, 108, 1993, p. 73.

Polymer compounds that can be used in the composition of the invention may also be selected from polymers that are grafted by a (C₂-C₅)alkyleneimine, preferably polymers that are grafted by ethyleneimine, more preferably polyamidoamines, that are crosslinked or not, grafted by the ethyleneimine.

Polyamidoamines may be prepared from polyalkylene polyamines selected from those having from 3 to 10 carbon atoms, as for example diethylene triamine, triethylene tetramine, dipropylene triamine, tripropylene tetramine, di-hexa methylene triamine, amino propylethylene diamine, bis-aminopropylethylene diamine, as well as combinations thereof, and from a monocarboxylic aliphatic acid.

As explained hereabove, the polymer compounds contained in the composition of the invention may also be selected from polycondensates containing at least one compound selected from a) piperazine, 1-(2-aminoethyl)piperazine, 1,4-bis(3-aminopropyl)piperazine), (C₁-C₂₅)1-alkyl piperazine, 1,4-di((C₁-C₂₅)alkyl)piperazine, 1-(2-hydroxy((C₂-C₂₅)alkyl))piperazine, imidazole, C₁-C₂₅ alkyl imidazole, or combinations thereof, with at least one compound selected from b) a C₆-C₂₂ dihalide alkylene, an epihalohydrine and/or a C₈-C₂₂ bis-epoxide.

Advantageously, the a) group component(s) and the b) group component(s) is or are present in a molar ratio ranging from 1:0.8 to 1:1.1.

A preferred polycondensate is for example the polycondensate from piperazine and imidazole with epihalohydrine.

Such polycondensates that can be used in the composition of the invention are inter alia described in the U.S. Pat. No. 6,025,322.

The polymer compounds that can be used in the composition of the invention may also be selected from polymers containing at least 2 units of one or more basic amino acids.

Said basic amino acid(s) is or are selected preferably from ornithin, asparagine, glutamine, lysine and arginine.

Said polymer compounds containing at least 2 units of one or more basic amino acids that can be used in the composition of the invention do generally contain from 2 to 10000 basic amino acid units.

The polymer compounds contained in the composition of the invention may further be selected from dendrimers containing end-positioned primary amines.

As used herein, “the dendrimers containing end-positioned primary amines” mean polymer compounds comprising a core and generations of basic units, monomers or spindles, onto which one T-terminal group carrying a primary amine functionality has been grafted.

Suitable examples thereof include the polyamidoamine type dendrimers, such as those marketed under the trade name STARBURST PAMAM by DENDRITECH (block copolymers of ethylene diamine and methyl acrylate), or those marketed under the trade name ASTROMOLS (DAB) by DSM.

As explained hereabove, the polymer compounds used in the composition of the invention are modified with one or more hydrocarbon hydrophilic and/or hydrophobic segments, said segment(s) being different from sugar and being devoid of any sulfur, silicone or amidino group, wherein the modification with one hydrophobic hydrocarbon segment is not carried out by means of a bifunctional spacer group.

Preferably, the hydrophilic segment(s) is or are selected from:

the segments of polyhydroxylated compounds, preferably polyalkylene glycol or polyvinyl alcohol segments,

the segments of polycarboxylated compounds as for example polyacrylic acids;

Polyalkylene glycol segments are generally selected from polyethylene glycol or polypropylene glycol segments.

Moreover, the hydrophobic segment(s) is or are preferably selected from fatty carbon chains.

Fatty carbon chains are selected from C₁₀-C₅₀ alkyl radicals, C₁₀-C₅₀ hydroxyalkyl radicals, C₁₀-C₅₀ carboxyalkyl radicals, ((C₁-C₁₀)alkoxy)carbonyl ((C₁₀-C₅₀)alkyl) radicals and C₁₂-C₅₀fatty acid esters.

Thus the modified polymer compounds that can be used in the composition of the invention may be selected from polyethyleneimine-polyethylene glycol, as for example compounds marketed by BASF under the trade names LUPASOL SC61B and SC62J, or by ALDRICH under the trade names 42347-5 and 30618-5.

The modified polymer compounds that can be used in the composition of the invention may further be selected from polyethyleneimine-polyvinyl alcohol, polyallylamine-polyethylene glycol and polyallylamine-polyvinyl alcohol, especially based on the polyallylamines marketed by NITTO BOSEKI Co.

They may further be selected from polylysine-polyethylene glycol and polylysine-polyvinyl alcohol, especially poly-ε-lysine-polyethylene glycol and poly-ε-lysine-polyvinyl alcohol, for example based on the poly-ε-lysine marketed by CHISSO.

They finally may be selected from fatty chain-containing polyethyleneimines amidified by fatty acids, for example the compound marketed by BASF under the trade name LUPASOL ESA.

The hydrophilic and/or hydrophobic segments may be graft onto the polymer compound(s), giving in that case a comb polymer. They also may be sequenced with the amine units.

The description of a synthetic route for alkoxylated basic poly(amino acids), especially for polylysine-polyethylene glycol can be found for example in the international patent application WO 00/71601.

The description of a synthetic route for polylysine-polyethylene glycol can also be found in the Journal of Physical Chemistry B, 2000, 104, p. 3298, G. L. Kenausis and al.

The description of a synthetic route for polyethyleneimine-polyethylene glycol can be found for example in the international patent application WO 97/20879, or also in Macromolecules, 2002, 35, p. 6867, H. Petersen and al.

The modified polymer compound( )s represent(s) advantageously from 0.01 to 40%, preferably from 0.1 to 20%, more preferably from 1 to 10%, by weight, as related to the composition total weight.

In addition to one or more modified polymer compound(s) such as those previously described, the composition of the invention advantageously may further comprise at least one cosmetic active agent selected from conditioning agents and styling agents other than the polyamines of the invention.

The conditioning agents are generally selected from cationic polymers, volatile or non volatile, linear or cyclic silicones, and silicone derivatives.

As used herein, “cationic polymer” means any polymer containing cationic groups and/or groups that are ionizable into cationic groups.

Suitable cationic polymers include more particularly polymers of polyamine, polyaminoamide and quaternary polyammonium type.

Polyamine, polyaminoamide, quaternary polyammonium type polymers, that can be used in the composition of the invention, are described inter alia in the French patents FR 2 505 348 and FR 2 542 997. Such polymers include:

(1) homopolymers or copolymers derived from acrylic or methacrylic acid esters or amides;

(2) cellulose ether derivatives comprising quaternary ammonium groups such as those described in the French patent FR 1 492 597;

(3) cationic cellulose derivatives such as cellulose copolymers or cellulose derivatives grafted with a water-soluble quaternary ammonium monomer, and those described in particular in the U.S. Pat. No. 4,131,576, such as hydroxyalkylcelluloses, such as hydroxymethyl-, hydroxyethyl- or hydroxypropyl-celluloses grafted especially with a methacryloylethyl trimethylammonium, methacrylamidopropyl trimethylammonium or dimethyl diallylammonium salt; polyquaternium 10 (INCI name) can be mentioned as a suitable example thereof;

(4) other cationic polysaccharides that are more particularly described in the U.S. Pat. Nos. 3,589,578 and 4,031,307 such as guar gums containing trialkylammonium cationic groups;

(5) polymers containing piperazinyl units and alkylene or hydroxyalkylene divalent groups with straight or branched chains, optionally interrupted by oxygen, sulfur, nitrogen atoms or by aromatic or heterocyclic rings, as well as oxidation and/or quaternization products of these polymers. Such polymers are inter alia described in the French patents FR 2 162 025 and FR 2 280 361;

(6) water-soluble polyaminoamides, such as those described in particular in the French patents FR 2 252 840 and FR 2 368 508;

(7) polyaminoamides derivatives, for example, polymers of adipic acid and dialkylaminohydroxyalkyldialkylene triamine wherein the alkyl group contains from 1 to 4 carbon atoms and is preferably a methyl, ethyl, propyl group, and wherein the alkylene group contains from 1 to 4 carbon atoms, and is preferably an ethylene group. Such polymers are especially described in the French patent FR 1 583 363;

(8) polymers that are obtained by reacting a polyalkylene polyamine having two primary amine groups and at least one secondary amine group, with a dicarboxylic acid selected from diglycolic acid and saturated, aliphatic dicarboxylic acids having from 3 to 8 carbon atoms. The molar ratio between the polyalkylene polyamine and the dicarboxylic acid ranging from 0.8:1 to 1.4:1; the resulting polyaminoamide being reacted with the epichlorhydrine in a molar ratio between the epichlorhydrine and the secondary amine group of the polyaminoamide ranging from 0.5:1 and 1.8:1. Such polymers are especially described in the U.S. Pat. Nos. 3,227,615 and 2,961,347;

(9) alkyldiallylamine or dialkyldiallylammonium cyclopolymers such as the dimethyldiallyl ammonium chloride homopolymer and the copolymers of diallyldimethylammonium chloride and acrylamide;

(10) quaternary diammonium polymers having a number average molecular weight generally ranging from 1000 to 100000, such as those described, for example, in the French patents FR 2 320 330, 2 270 846, 2 316 271, 2 336 434 and 2 413 907 and in the U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020; hexadimethrine chloride (INCI name), marketed by CHIMEX under the trade name MEXOMERE PO may be mentioned as an example thereof;

(11) quaternary polyammonium polymers such as those especially described in the European patent application EP-A-122 324;

(12) vinylpyrrolidone and vinylimidazole quaternary polymers such as, for example, the products marketed under the trade names Luviquat® FC 905, FC 550 and FC 370 by BASF;

(13) polyamines such as Polyquart® H marketed by HENKEL, referenced to as “POLYETHYLENE GLYCOL (15) TALLOW POLYAMINE” in the CTFA dictionary;

(14) crosslinked polymers based on methacryloyloxy(C₁-C₄)alkyl (C₁-C₄)trialkylammonium salts such as those marketed under the trade names SALCARE® SC 92, SALCARE® SC 95 and SALCARE® SC 96 by ALLIED COLLOIDS; and combinations thereof.

Other cationic polymers that can be used within the scope of the present invention are cationic proteins or cationic protein hydrolysates, polyalkyleneimines, especially polyethyleneimines, vinylpyridine or vinylpyridinium unit-containing polymers, polyamine and epichlorhydrine condensates, quaternary polyureylenes and chitin derivatives.

The cationic polymers are preferably selected from hexadimethrine chloride and dimethyldiallylammonium chloride homo- or copolymers.

As explained hereabove, the conditioning agents may also be selected from silicones.

Silicones that can be suitably used as cosmetic active agents in the method according to the invention include polydimethylsiloxanes, quaternized polyorganosiloxanes such as those described in the French patent application FR 2 535 730, aminoalkyl group-containing polyorganosiloxanes modified with alkoxycarbonylalkyl groups such as those described in the U.S. Pat. No. 4,749,732, polyorganosiloxanes such as the copolymer of polydimethylsiloxane and polyoxyalkyl of the dimethicone copolyol type, a polydimethylsiloxane having stearoxy end groups (stearoxydimethicone), a copolymer of polydimethylsiloxane and dialkylammonium acetate or a copolymer of polydimethylsiloxane and polyalkylbetaine described in the French patent FR 2 197 352, polysiloxanes organomodified with mercapto or mercaptoalkyl groups such as those described in the French patent FR 1 530 369 and in the European patent application EP 295 780.

As explained hereabove, in addition to the conditioning agents, the composition of the invention may advantageously contain one or more styling agents.

The styling agents are generally selected from amphoteric, anionic or non ionic polymers.

Amphoteric polymers that can be used as styling agents according to the invention may be selected from polymers having B and C units randomly distributed within the polymer chain, wherein B represents a unit derived from a monomer containing at least one basic nitrogen atom and C represents a unit derived from an acid monomer containing one or more carboxylic or sulfonic groups, or B and C may represent groups derived from carboxybetaine or sulfobetaine zwitterionic monomers; B and C may also represent a cationic polymer chain containing primary, secondary, tertiary or quaternary amine groups, wherein at least one of the amine groups is carrying a carboxylic or a sulfonic group linked through a hydrocarbon group, or B and C belong to a polymer chain containing an dicarboxylic ethylene unit, one of the carboxylic groups of which was reacted with a polyamine containing one or more primary amine or secondary amine groups.

Amphoteric polymers according to the above definition that are more particularly preferred are selected from the following polymers:

(1) polymers resulting from the copolymerization of a monomer derived from a vinyl compound carrying a carboxylic group such as more particularly acrylic acid, methacrylic acid, maleic acid, alpha-chloracrylic acid, and of a basic monomer derived from a substituted vinyl compound containing at least one basic atom such as more particularly dialkylaminoalkyl-methacrylates and acrylates, dialkylaminoalkyl-methacrylamides and acrylamides. Such compounds are described in the U.S. Pat. No. 3,836,537. The copolymer of sodium acrylate and acrylamidopropyl trimethylammonium chloride marketed under the trade name POLYQUART® KE 3033 by HENKEL may also be mentioned as a suitable example.

The vinyl compound may also be a dialkyldiallylammonium salt such as the diethyldiallyl ammonium chloride.

Copolymers made of acrylic acid and of this later monomer are marketed under the trade names MERQUAT® 280, MERQUAT® 295 and MERQUAT® PLUS 3330 by CALGON.

(2) Polymers comprising units derived from:

a) at least one monomer selected from acrylamides or methacrylamides substituted on the nitrogen by an alkyl group,

b) at least one acid comonomer containing one or more carboxylic reactive groups, and

c) at least one basic comonomer such as acrylic and methacrylic acid esters with primary, secondary, tertiary and quaternary amine substituants, and the quaternization product of dimethylaminoethyl methacrylate with dimethyl or diethyl-sulfate.

N-substituted acrylamides or methacrylamides that are more particularly preferred according to the invention are the groups the alkyl groups of which do contain from 2 to 12 carbon atoms and more particularly N-ethylacrylamide, N-tertiobutylacrylamide, N-tertiooctylacrylamide, N-octylacrylamide, N-decylacrylamide, N-dodecylacrylamide as well as corresponding methacrylamides.

Acid comonomers are selected more particularly from acrylic, methacrylic, crotonic, itaconic, maleic, fumaric acids as well as alkyl monoesters containing from 1 to 4 carbon atoms of the maleic or fumaric acids or anhydrides.

The preferred basic comonomers are aminoethyl, butylaminoethyl, N,N′-dimethylaminoethyl, N-tertio-butylaminoethyl methacrylates.

Particularly employed are copolymers which CTFA name (fourth Ed, 1991) is octylacrylamide/acrylates/butylaminoethyl-methacrylate copolymer such as the products marketed under the trade name AMPHOMER® or LOVOCRYL® 47 by NATIONAL STARCH.

(3) Crosslinked and partly or fully alkylated polyaminoamides derived from polyaminoamides having following general formula:

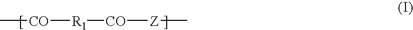

wherein R₁ represents a divalent group derived from a saturated dicarboxylic acid, an aliphatic mono- or dicarboxylic acid with a double ethylene bond, an ester of a lower alkanol having from 1 to 6 carbon atoms of these acids or of a group resulting from the addition of any of said acids with a bis-primary or a bis-secondary amine, and Z represents a group of a bis-primary, mono or bis-secondary polyalkylene-polyamine and represents preferably:

a) in amounts ranging from 60 to 100 mol %, the following group

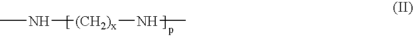

wherein x=2 and p=2 or 3, or x=3 and p=2

this group being derived from diethylene-triamine, triethylene-tetraamine or dipropylene-triamine;

b) in amounts ranging from 0 to 40 mol %, the hereabove group (II), wherein x=2 and p=1 and that is derived from ethylenediamine, or the group derived from piperazine

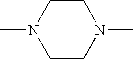

c) in amounts ranging from 0 to 20 mol %, the group —NH—(CH₂)₆—NH— derived from hexamethylenediamine, these polyaminoamines being crosslinked by adding a bifunctional crosslinking agent selected from epihalohydrines, diepoxides, dianhydrides, unsaturated bis derivatives, using from 0.025 to 0.35 mol of a crosslinking agent for each amine group of the polyaminoamide and alkylated by means of acrylic acid, chloracetic acid or an alkane-sultone or salts thereof.

Carboxylic or dicarboxylic acids that can be used as R₁ are preferably selected from acids having from 6 to 10 carbon atoms such as adipic, 2,2,4-trimethyladipic and 2,4,4-trimethyladipic, terephthalic acids and ethylene double bond acids such as, for example, acrylic, methacrylic and itaconic acids.

The alkane-sultones that are used for alkylation are preferably propane- or butane-sultone. Alkylating agent salts are preferably sodium or potassium salts.

(4) Polymers containing zwitterionic units having following formula:

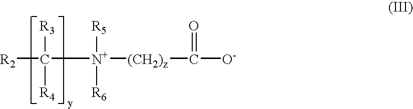

wherein R₂ represents an unsaturated polymerizable group such as an acrylate, methacrylate, acrylamide or methacrylamide group, y and z each represent an integer ranging from 1 to 3, R₃ and R₄ represent a hydrogen atom, a methyl, ethyl or propyl group, R₅ and R₆ represent a hydrogen atom or an alkyl group so that the sum of the carbon atoms in R₅ and R₆ does not exceed 10.

Polymers comprising such units may also contain units derived from non zwitterionic monomers such as dimethyl- or diethylaminoethyl acrylate or methacrylate, or alkyl acrylates or methacrylates, acrylamides or methacrylamides, or vinyl acetate.

Suitable examples include the copolymer of methyl methacrylate and methyl dimethyl-carboxymethylammonioethylmethacrylate such as the product marketed under the trade name DIAFORMER® Z301 by SANDOZ.

(5) Chitosan-derived polymers comprising monomer units having following formulas:

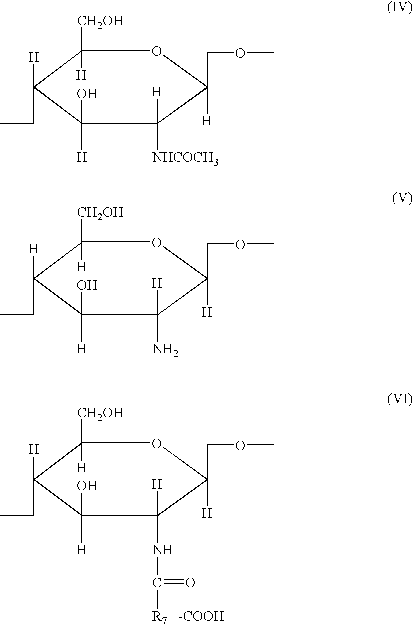

the (IV) unit being present in amounts ranging from 0 to 30%, the (V) unit in amounts ranging from 5 to 50% and the (VI) unit in amounts ranging from 30 to 90%, being understood that in this (VI) unit, R₇ represents a group having following formula:

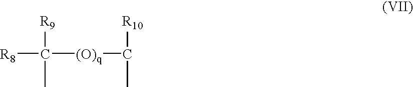

wherein if q=0, R₈, R₉ and R₁₀, being the same or different, each represent a hydrogen atom, a methyl, hydroxyl, acetoxy or amino radical, a monoalkylamine radical or a dialkylamine radical optionally interrupted by one or more nitrogen atoms and/or optionally substituted by one or more amine, hydroxyl, carboxyl, alkylthio, sulfonic groups, an alkylthio radical which alkyl group carries an amino radical, at least one of the R₈, R₉ and R₁₀ groups being in that case a hydrogen atom;

or if q=1, R₈, R₉ and R₁₀ each represent a hydrogen atom, as well as salts formed by these compounds with bases or acids.

(6) polymers resulting from the chitosan N-carboxyalkylation such as the N-carboxymethyl-chitosan or the N-carboxybutyl-chitosan marketed under the trade name EVALSAN® by JAN DEKKER.

(7) polymers having following general formula (VIII) described for example, in the French patent FR 1 400 366:

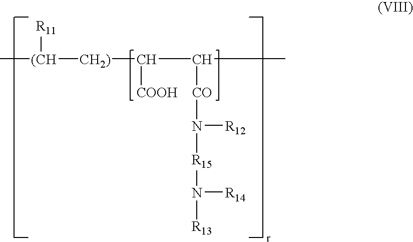

wherein R₁₁ represents a hydrogen atom, a CH₃O, CH₃CH₂O, or a phenyl group, R₁₂ represents hydrogen or a lower alkyl group such as methyl, ethyl, R₁₃ represents hydrogen or a lower alkyl group such as methyl, ethyl, R₁₄ represents a lower alkyl group such as methyl, ethyl or a group having following formula: —R₁₅—N(R₁₃)₂, R₁₅ being a —CH₂—CH₂—, —CH₂—CH₂—CH₂—, —CH₂—CH(CH₃)— group, R₁₃ being as defined hereabove,

as well as the higher homologues of these groups and containing up to 6 carbon atoms.

(8) amphoteric polymers of -D-X-D-X- type selected from:

a) polymers obtained by the action of chloracetic acid or sodium chloracetate on compounds comprising at least one unit having following formula:
-D-X-D-X-D- (IX)

wherein D represents a group

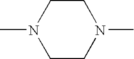

and X represents the E or E′ symbol, E or E′ being the same or different, representing a bivalent group that is an alkylene group having a straight or a branched chain comprising up to 7 carbon atoms in the main chain not substituted or substituted by hydroxyl groups, and comprising optionally some oxygen, nitrogen, sulfur atoms, from 1 to 3 aromatic and/or heterocyclic rings, oxygen, nitrogen and sulfur atoms being present in the form of ether, thioether, sulfoxide, sulfone, sulfonium, alkylamine, alkenylamine, hydroxyl, benzylamine, amine oxide, quaternary ammonium, amide, imide, alcohol, ester and/or urethane groups.

b) Polymers having following formula:
-D-X-D-X- (X)

wherein D represents a group

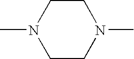

and X represents the E or E″ symbol and at least one time E″; E being as defined hereabove and E″ is a bivalent group that is an alkylene group with a straight or branched chain having up to 7 carbon atoms in the main chain, substituted or not by one or more hydroxyl groups and comprising one or more nitrogen atoms, the nitrogen atom being substituted by an alkyl chain optionally interrupted by one oxygen atom and comprising compulsorily one or more carboxyl functionalities or one or more hydroxyl functionalities betainized upon reaction with chloracetic acid or sodium chloracetate.

(9) copolymers of (C₁-C₅)alkylvinylether and maleic anhydride partially modified by half-amidification with a N,N-dialkylaminoalkylamine such as N,N-dimethylaminopropyl-amine or by half-esterification with a N,N-dialkanolamine. These copolymers may also contain other vinyl comonomers such as vinyl caprolactame.

The styling agent(s) may further be selected from anionic polymers.

The anionic polymers generally used in this invention are polymers comprising groups derived from carboxylic, sulfonic or phosphoric acids, and having a weight molecular weight ranging from 500 to 5 000 000.

The carboxylic groups are provided by unsaturated monoacid or diacid carboxylic monomers such as those having following formula: 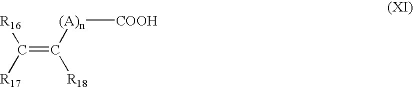
wherein n is an integer ranging from 0 to 10, A represents a methylene group, optionally linked to the unsaturated group carbon atom or to the adjacent methylene group when n is more than 1, through a heteroatom such as oxygen or sulfur, R₁₆ represents a hydrogen atom, a phenyl or a benzyl group, R₁₇ represents a hydrogen atom, a lower alkyl group or a carboxyl group, R₁₈ represents a hydrogen atom, a lower alkyl group, a —CH₂—COOH, phenyl or benzyl group.

In the above formula (XI), the lower alkyl group(s) preferably contain(s) from 1 to 4 carbon atoms and represent(s), especially, methyl and ethyl groups.

Anionic polymers with carboxylic groups preferred according to the invention include:

A) acrylic or methacrylic acid homo- or copolymers or their salts, and especially the products marketed under the trade names VERSICOL® E or K by ALLIED COLLOID, ULTRAHOLD® by BASF, copolymers of acrylic acid and acrylamide marketed as their sodium salt under the trade names RETEN® 421, 423 or 425 by HERCULES, sodium salts of polyhydroxycarboxylic acids.

B) acrylic or methacrylic acid copolymers with a monoethylene monomer such as ethylene, styrene, vinyl esters, acrylic or methacrylic acid esters, optionally grafted onto a polyalkylene glycol such as polyethylene glycol, and optionally crosslinked. Such polymers are especially described in the French patent FR 1 222 944 and in the German application n^{o} 2 330 956, the copolymers of this type comprising in their chain an optionally N-alkylated and/or hydroxyalkylated acrylamide unit, such as those described in particular in the Luxemburger patent applications n^{o} 75370 and 75371 or provided under the trade name QUADRAMER® by AMERICAN CYANAMID. Copolymers of acrylic acid and C₁-C₄ alkyl methacrylate and the copolymer of methacrylic acid and ethyl acrylate marketed under the trade name LUVIMER® MAEX by BASF may also be mentioned.

C) copolymers derived from crotonic acid such as those comprising in their chain vinyl acetate or vinyl propionate units, and optionally other monomers such as allyl or methallyl esters, vinyl ethers or vinyl esters of a saturated, linear or branched, long hydrocarbon chain-carboxylic acid, such as those comprising at least 5 carbon atoms, these polymers being optionally graft and crosslinked, or a vinyl, allyl or methallyl ester of a carboxylic α or β-cyclic acid. Such polymers are described inter alia in the French patents n^{o} FR 1 222 944, FR 1 580 545, FR 2 265 782, FR 2 265 781, FR 1 564 110 and FR 2 439 798. Such commercial products belonging to this class are for example the 28-29-30, 26-13-14 and 28-13-10 resins marketed by NATIONAL STARCH.

D) polymers derived from maleic, fumaric, itaconic acids or anhydrides with vinyl esters, vinyl ethers, vinyl halides, phenylvinyl derivatives, acrylic acid and esters thereof. These polymers may be esterified. Such polymers are especially described in the U.S. Pat. Nos. 2,047,398, 2,723,248, 2,102,113, and in the British patent GB 839 805. Those marketed under the trade names GANTREZ® AN or ES by ISP may be especially mentioned.

Polymers also belonging to this class are copolymers of maleic, citraconic, itaconic anhydrides and of an allylic or methallyl ester optionally comprising an acrylamide, methacrylamide group, an a-olefin, acrylic or methacrylic esters, acrylic or methacrylic acids or vinylpyrrolidone in their chain, wherein their anhydride functionalities are monoesterified or monoamidified. These polymers are for example described in the French patents FR 2 350 384 and FR 2 357 241 of the applicant.

E) polyacrylamides comprising carboxylate groups.

As explained hereabove, anionic polymers may also be polymers comprising sulfonic acid derived groups.

Polymers comprising sulfonic groups are polymers comprising vinyl sulfonic, styrene sulfonic, naphthalene sulfonic or acrylamido alkylsulfonic units.

These polymers may especially be selected from:

polyvinylsulfonic acid salts having a molecular weight ranging from about 1 000 to 100 000 as well as copolymers with an unsaturated comonomer such as acrylic or methacrylic acids, and esters thereof, as well as acrylamide or its derivatives, vinyl ethers and vinylpyrrolidone;

polystyrene sulfonic acid salts, especially sodium salts, having a molecular weight of about 500 000 and of about 100 000 respectively marketed under the trade names Flexan® 500 and Flexan® 130 by National Starch. These compounds are described in the French patent FR 2 198 719;

polyacrylamide sulfonic acid salts such as those mentioned in the U.S. Pat. No. 4,128,631, and more particularly the polyacrylamidoethylpropane sulfonic acid marketed under the trade name COSMEDIA POLYMER® HSP 1180 by Henkel.

Finally, the styling agents may be selected from non ionic polymers.

Non ionic polymers that can be used according to the present invention especially include:

vinylpyrrolidone homopolymers;

copolymers of vinylpyrrolidone and vinyl acetate;

polyalkyloxazolines such as polyethyloxazolines marketed by DOW CHEMICAL under the trade names PEOX® 50 000, PEOX® 200 000 and PEOX® 500 000;

vinyl acetate homopolymers such as the product marketed under the trade name APPRETAN® EM by HOECHST or the product marketed under the trade name RHODOPAS® A 012 by RHONE POULENC;

copolymers of vinyl acetate and acrylic ester such as the product marketed under the trade name RHODOPAS® AD 310 de RHONE POULENC;

copolymers of vinyl acetate and ethylene such as the product marketed under the trade name APPRETAN® TV by HOECHST;

copolymers of vinyl acetate and maleic ester, for example dibutyl maleate such as the product marketed under the trade name APPRETAN® MB EXTRA by HOECHST;

copolymers of polyethylene and maleic anhydride;

alkyl acrylate homopolymers and alkyl methacrylate homopolymers such as the product marketed under the trade name MICROPEARL® RQ 750 by MATSUMOTO or the product marketed under the trade name LUHYDRAN® A 848 S by BASF;

acrylic ester copolymers such as, for example, copolymers of alkyl acrylates and alkyl methacrylates such as the products marketed by ROHM & HAAS under the trade names PRIMAL® AC-261 K and EUDRAGIT® NE 30 D, by BASF under the trade names ACRONAL® 601, LUHYDRAN® LR 8833 or 8845, by HOECHST under the trade names APPRETAN® N 9213 or N921 2;

copolymers of acrylonitrile with a non ionic monomer selected, for example, from butadiene and alkyl (meth)acrylates; the products marketed under the trade names NIPOL® LX 531 8 by NIPPON ZEON may be mentioned or those marketed under the trade name CJ 0601 8 by ROHM & HAAS;

copolymers of alkyl acetate and urethane such as the product 8538-33 by NATIONAL STARCH;

polyamides such as the product ESTAPOR® LO 11 marketed by RHONE POULENC;

non ionic guar gums, that have been chemically modified or not.

Non modified, non ionic guar gums are, for example, the products marketed under the trade name VIDOGUM® GH 175 by UNIPECTINE and under the trade name JAGUAR® C by MEYHALL.

Modified, non ionic guar gums, that can be used according to the invention, are preferably modified with C₁-C₆ hydroxyalkyl groups. Hydroxymethyl, hydroxyethyl, hydroxypropyl and hydroxybutyl groups may be mentioned as suitable examples.

These guar gums are well known from the state of the art and may be prepared, for example, by reacting corresponding alkene oxides, such as, for example, propylene oxides, with the guar gum so as to obtain a guar gum modified with hydroxypropyl groups.

Such non ionic guar gums that are optionally modified with hydroxyalkyl groups are, for example, marketed under the trade names JAGUAR® HP8, JAGUAR® HP60 and JAGUAR® HP120, JAGUAR® DC 293 and JAGUAR® HP 105 by MEYHALL, or under the trade name GALACTASOL® 4H4FD2 by AQUALON.

The alkyl groups of the non ionic polymers contain preferably from 1 to 6 carbon atoms.

The conditioning agent(s) and/or the styling agent(s) represent generally from 0.01 to 40%, preferably from 0.1 to 20%, by weight as related to the total weight of the composition of the invention.

In addition to the conditioning agents and to the styling agents, the composition of the invention may comprise at least one further cosmetic active agent selected from inorganic or organic, associative or not, gelling agents and/or thickening agents, anionic, non ionic, cationic or amphoteric surfactants, propenetrating agents, emulsifying agents, fragrances, preservatives, fillers, sunscreens, coloring agents, proteins, vitamins, provitamins, moisturizing agents, emollients, softening agents, mineral, vegetal or synthetic oils, hydrophilic or lipophilic active agents such as ceramides and pseudoceramides, antifoaming agents, antiperspirants, free radical scavengers, bactericides, and anti-dandruff agents.

The components of the composition of the invention are advantageously included in a cosmetically acceptable medium.

Advantageously, the cosmetically acceptable medium comprises at least one solvent selected from water, C₂-C₆ alcohols, C₂-C₆ ethers, C₂-C₆ esters, N-methylpyrrolidone (NMP), C₃-C₆ ketones, polyols, and polyol ethers or esters.

The composition of the invention may come as a hair-care lotion, a shampoo or a hair cream rinse, a scalp-cleaning liquid or solid soap, a hair shaping product (hair spray, hair setting product, styling gel), a hair treating masque, a hair cream or a hair foaming gel. It also may be in the form of a hair dying product or a hair mascara, of hair wave products for permanenting or uncurling the hair.

The composition of the invention may be conditioned in an aerosol device. In this case, it comprises at least one propellant, that may be selected from volatile hydrocarbons, such as n-butane, propane, isobutane, pentane, halogenated hydrocarbons and combinations thereof. Carbon dioxide, nitrogen protoxide, dimethylether (DME), nitrogen or compressed air may also be used as a propellant. Propellant combinations may also be used. Preferably dimethylether will be used.

Advantageously, the propellant is present in an amount ranging from 5 to 90% by weight as related to the total weight of the composition in the aerosol device, et, more particularly, in an amount ranging from 10 to 60%.

It is a further object of the invention to use for providing softness to the hair, a cosmetic composition containing in a cosmetically acceptable medium, at least one type of polymer compound whose chain comprises at least two types of amine units —NH— and/or 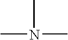
and is devoid of any vinyl amine or vinyl amide unit, said polymer compound being modified with one or more hydrophilic and/or hydrophobic hydrocarbon segments, said segment(s) being devoid of any sulfur, silicone or amidino group.

Preferably, the hydrophilic segment(s) is or are not sugars.

The invention also provides the use, as a cosmetic active agent evenly distributing onto the hair, of a polymer compound which chain comprises at least two amine units —NH— and/or 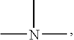
, said polymer compound being modified with one or more hydrophilic and/or hydrophobic hydrocarbon segments.

Finally, it is an object of the invention to provide the use, for improving the deposition homogeneity of at least one cosmetic active agent onto the keratinic materials, of a cosmetic composition containing, in a cosmetically acceptable medium, at least one type of polymer compound whose chain comprises at least two types of amine units —NH— and/or 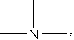
said polymer compound being modified with one or more hydrophilic and/or hydrophobic hydrocarbon segments.

The cosmetic active agent which deposition homogeneity is improved thanks to the method of the invention may be included in the composition or be applied onto the keratinic materials once the cosmetic composition has been applied.

When the cosmetic active agent is applied after the cosmetic composition application, it is advantageously anionic in nature.

Preferably, the cosmetic active agent which deposition homogeneity is improved is selected from conditioning agents and styling agents.

The conditioning agents and the styling agents are such as previously defined.

Thus, the conditioning agents may be selected from cationic polymers, volatile or non volatile, linear or cyclic silicones, and silicone derivatives.

In the same way, the styling agents may be selected from anionic, non ionic or amphoteric polymers.

The cosmetic active agent may also be selected from all conventional cosmetic active agents such as gelling agents and/or inorganic or organic, associative or not associative thickening agents, anionic, non ionic, cationic or amphoteric surfactants, propenetrating agents, emulsifying agents, fragrances, preservatives, fillers, sunscreens, coloring agents, proteins, vitamins, provitamins, moisturizing agents, emollients, softening agents, mineral, vegetal or synthetic oils, hydrophilic or lipophilic active agents such as ceramides and pseudoceramides, antifoaming agents, antiperspirants, free radical scavengers, bactericides, and anti-dandruff agents.

The polymer compounds used according to the invention may be those as previously defined as regards the composition of the invention.

However, moreover, when the polymer compounds are used for improving the uniform deposition of a cosmetic active agent onto the keratinic materials according to the invention, they may optionally contain one or more vinyl amine and/or vinyl amide units.

The hydrophilic and hydrophobic segments for modifying the polymer compounds used according to the invention may be those as previously defined as regards the composition of the invention.

Thus, the hydrophilic segments may be selected from:

segments of polyhydroxylated compounds, preferably polyalkylene glycol or polyvinyl alcohol segments, and

segments of polycarboxylated compounds.

In the same way as previously stated, polyalkylene glycol segments are preferably selected from polyethylene glycol or polypropylene glycol segments,

Moreover, as previously stated, hydrophobic segments are selected from fatty acid carbon chains.

The fatty acid carbon chains are preferably selected from C₁₂-C₅₀ fatty alcohols, C₁₂-C₅₀ fatty acids, and C₁₂-C₅₀ fatty acid esters.

Moreover, when the polymer compounds are used for improving the homogeneity of a cosmetic active agent onto the keratinic materials according to the invention, they may be modified with sugar segments or by silicone segments.

Sugars may be selected for example from polysaccharides. Glucose, galactose, maltose, cellobiose and maltotriose may be especially mentioned.

Silicone segments may be for example polydimethylsiloxane segments.

Thus, the modified polymer compounds used for improving the homogeneity of a cosmetic active agent onto the keratinic materials according to the invention may be selected for example from polyethyleneimine-polysaccharides, polyethyleneimine-mono, di and trisaccharides, such as polyethyleneimine-glucose, polyethyleneimine-galactose, polyethyleneimine-maltose, polyethyleneimine-cellobiose, and polyethyleneimine-maltotriose.

Moreover, as previously stated, the modified polymer compounds used according to the invention may be selected from polyethyleneimine-polyethylene glycol, polyethyleneimine-polyvinyl alcohol, polyallylamine-polyethylene glycol, polyallylamine-polyvinyl alcohol, polylysine-polyethylene glycol and polylysine-polyvinyl alcohol.

As previously stated, the modified polymer compound(s) represent from 0.01 to 40%, preferably from 0.1 to 20%, more preferably from 1 to 10%, by weight as related to the composition total weight.

When the composition is used for providing softness to the hair according to the invention, it may comprise any conventional cosmetic active agent.

The composition of the invention may be applied and be either rinsed off or not.

Once the composition of the invention has been applied, the hair may undergo a heating step which temperature is preferably higher than 30° C.

The application may be conducted either prior to or after any conventional cosmetic treatment. Hair coloring, permanent hair shaping, uncurling and straightening operations and shampoo processes may be especially mentioned.

This invention will be illustrated by following examples:

### EXAMPLE 1

### Modified polyethyleneimine Synthesis

A. Polyethyleneimine-glucose Synthesis

A 10% by weight polyethyleneimine aqueous A solution was prepared, using the polyethyleneimine marketed by BASF under the trade name LUPASOL P.

Its pH value was adjusted to 8.5 by adding a necessary amount of hydrochloric acid 6N.

A 10% by weight glucose aqueous B solution was prepared at the same time.

Under stirring and at ambient temperature (about 20° C.), the A and B solutions were mixed together, to which 10 mL were added from a 3.5% by weight sodium cyanoborohydride NaBH₃CN aqueous solution.

Stirring was maintained for 12 hours.

A polyethyleneimine-glucose polymer was obtained, which was then purified by means of an exclusion chromatography.

This polymer could then be used according to the invention.

B. Polyethyleneimine-polyethylene Glycol Synthesis

A 5% by weight polyethyleneimine aqueous C solution was prepared, using the polyethyleneimine marketed by BASF under the trade name LUPASOL P.

Its pH value was adjusted to 8.5 by adding a necessary amount of hydrochloric acid 6N and a borate buffer.

Under stirring and at ambient temperature (about 20° C.), 2.16 g of a polyethene glycol compound containing an activated ester, o[(N-succinimidyl)succinyl]-o′-methyl-polyethylene glycol, marketed by FLUKA under the trade name 85976 were added to the C solution.

Stirring was maintained for 6 hours.

A polyethyleneimine-polyethylene glycol polymer was thus obtained, which was then purified by means of a dialysis.

This polymer could then be used according to the invention.

### EXAMPLE 2

### Application Onto the Hair of a Polymer Compound Modified According to the Invention

The two following compositions were prepared:

Composition 1: polyethyleneimine-Containing Composition

LUPASOL P (50% active material): 10 g
Water: q.s. 100 g
   Composition 2: Polyethyleneimine-polyethylene Glycol-Containing Composition
ALDRICH 42347-5: 5 g
Water: q.s. 100 g

Compositions 1 and 2 were each applied onto different clean hair streaks.

Streaks were placed under 40° C. for 30 minutes, then rinsed with water.

1. Effect on Hair Softness

The streaks treated with composition 1 were rigid and a sticky and very heavy feel appeared after 3 shampoos.

After 10 shampoos, the polymer deposit still remained but the streaks felt overloaded and heavy.

The streaks that had been treated with composition 2 felt slippery and slightly set.

After 3 shampoos, the streaks felt soft and coated. After 10 shampoos, the polymer deposit still remained and the streaks felt soft and smooth.

2. Effect on the Deposition Homogeneity of a Coloring Material

The effect of the application of compositions 1 and 2 on the deposition homogeneity of a coloring agent was tested.

After application of compositions 1 and 2 as previously defined each streak was colored using an anionic coloring agent Red 80.

The streaks that had been previously treated with composition 1, then colored with Red 80 resulted in an uneven coloration, that is to say there were colored areas and uncolored areas.

The streaks that had been previously treated with composition 2, then colored with Red 80 resulted in a uniform coloration.

1. An aqueous cosmetic composition containing, in a cosmetically acceptable medium, at least one type of polymer compound whose chain comprises at least two types of amine units —NH— and/or 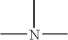
and is devoid of any vinyl amine or vinyl amide unit, said polymer compound being modified with one or more hydrophilic and/or hydrophobic hydrocarbon segments, said segment(s) being different from sugar and being devoid of any sulfur, silicone or amidino group, wherein the modification with hydrophobic hydrocarbon segments is not carried out by means of a bifunctional spacer group.

2. A composition according to claim 1, characterized in that the polymer compound(s) is or are selected from:
polyalkyleneimines, preferably poly((C₂-C₅)alkyleneimines),
polymers that are grafted by a (C₂-C₅)alkyleneimine, preferably polymers that are grafted by an ethyleneimine, more preferably polyamidoamines, crosslinked or not, grafted by the ethyleneimine,
copolymers based on amino (C₁-C₄)alkyl(meth)acrylate, preferably based on aminoethyl(meth)acrylate,
polyallylamines,
polycondensates of at least one compound selected from piperazine, 1-(2-aminoethyl)piperazine, 1,4-bis(3-aminopropyl)piperazine), 1-(C₁-C₂₅) alkyl piperazine, 1,4-di((C₁-C₂₅)alkyl)piperazine, 1-(2-hydroxy((C₂-C₂₅)alkyl))piperazine, imidazole, C₁-C₂₅ alkylimidazole, or combinations thereof, with at least one compound selected from a C₆-C₂₂ alkylene dihalide, an epihalohydrine and/or a C₈-C₂₂ bisepoxide,
polymers containing at least 2 units of one or more basic amino acids, and
dendrimers containing end-positioned primary amines.

3. A composition according to claim 2, characterized in that basic amino acid(s) is or are selected from ornithin, asparagine, glutamine, lysine and arginine.

4. A composition according to anyone of preceding claims, characterized in that the polymer compound(s) is or are linear, branched, hyper-branched or dendrimeric.

5. A composition according to anyone of preceding claims, characterized in that the hydrophilic segment(s) is or are selected from:
segments of polyhydroxylated compounds, preferably polyalkylene glycol or polyvinyl alcohol segments, and
segments of polycarboxylated compounds.

6. A composition according to claim 5, characterized in that the polyalkylene glycol segments are selected from polyethylene glycol or polypropylene glycol segments.

7. A composition according to anyone of preceding claims, characterized in that the hydrophobic segment(s) is or are selected from fatty acid carbon chains.

8. A composition according to claim 7, characterized in that the fatty acid carbon chains are selected from C₁₀-C₅₀ alkyl radicals, C₁₀-C₅₀ hydroxyalkyl radicals, C₁₀-C₅₀ carboxyalkyl radicals, ((C₁-C₁₀)alkoxy)carbonyl ((C₁₀-C₅₀) alkyl) radicals, C₁₂-C₅₀ fatty acid esters.

9. A composition according to anyone of preceding claims, characterized in that the hydrophobic and/or hydrophilic segment(s) is or are grafted onto the polymer compound(s) or sequenced with the amine units.

10. A composition according to anyone of preceding claims, characterized in that the modified polymer compound(s) is or are selected from polyethyleneimine-polyethylene glycol, polyethyleneimine-polyvinyl alcohol, polyallylamine-polyethylene glycol, polyallylamine-polyvinyl alcohol, polylysine-polyethylene glycol and polylysine-polyvinyl alcohol.

11. A composition according to anyone of preceding claims, characterized in that the modified polymer compound(s) represent(s) from 0.01 to 40%, preferably from 0.1 to 20%, more preferably from 1 to 10%, by weight as related to the composition total weight.

12. A composition according to anyone of preceding claims, characterized in that it comprises at least one cosmetic active agent selected from conditioning agents and styling agents.

13. A composition according to claim 12, characterized in that the conditioning agent(s) is or are selected from cationic polymers, volatile or non volatile, linear or cyclic silicones, and silicone derivatives.

14. A composition according to claim 12, characterized in that the styling agent(s) is or are selected from anionic, non ionic or amphoteric polymers.

15. A composition according to anyone of claims 1 1 to 13, characterized in that the conditioning agent(s) and/or the styling agent(s) represent from 0.01 to 40%, preferably from 0.1 to 20%, by weight as related to the composition total weight.

16. A composition according to anyone of preceding claims, characterized in that it comprises in addition at least one cosmetic active agent selected from gelling agents and/or inorganic or organic, associative or non associative thickening agents, anionic, non ionic, cationic or amphoteric surfactants, propenetrating agents, emulsifying agents, fragrances, preservatives, fillers, sunscreens, coloring agents, proteins, vitamins, provitamins, moisturizing agents, emollients, softening agents, mineral, vegetal or synthetic oils, hydrophilic or lipophilic active agents such as ceramides and pseudoceramides, antifoaming agents, antiperspirants, free radical scavengers, bactericides, and anti-dandruff agents.

17. A composition according to anyone of preceding claims, characterized in that it comprises at least one solvent selected from water, C₂-C₆ alcohols, C₂-C₆ ethers, C₂-C₆ esters, N-methylpyrrolidone (NMP), C₃-C₆ ketones, polyols, and polyol ethers or esters.

18. Use for providing softness to the hair, of a cosmetic composition that contains, in a cosmetically acceptable medium, at least one type of polymer compound whose chain comprises at least two types of amine units —NH— and/or 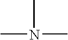
and is devoid of any vinyl amine or vinyl amide unit, said polymer compound being modified with one or more hydrophilic and/or hydrophobic hydrocarbon segments, said segment(s) being devoid of any sulfur, silicone or amidino group.

19. Use according to claim 18, characterized in that the hydrophilic segment(s) is or are not sugars.

20. Use as a cosmetic active agent evenly distributing onto the hair, of a polymer compound which chain comprises at least two amine units —NH— and/or 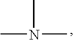
said polymer compound being modified with one or more hydrophilic and/or hydrophobic hydrocarbon segments.

21. Use for improving the deposition homogeneity of at least one cosmetic active agent onto the keratinic materials, of a cosmetic composition that contains, in a cosmetically acceptable medium, at least one type of polymer compound whose chain comprises at least two types of amine units —NH— and/or 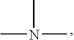
said polymer compound being modified with one or more hydrophilic and/or hydrophobic hydrocarbon segments.

22. Use according to claim 21, characterized in that the cosmetic active agent is included in the cosmetic composition or is applied onto the keratinic materials once the composition has been applied.

23. Use according to claim 21 or 22, characterized in that the cosmetic active agent is selected from conditioning agents and styling agents.

24. Use according to claim 23, characterized in that conditioning agents are selected from cationic polymers, volatile or non volatile, linear or cyclic silicones, and silicone derivatives.

25. Use according to claim 23, characterized in that the styling agents are selected from anionic, non ionic or amphoteric polymers.

26. Use according to claim 21 or 22, characterized in that the cosmetic active agent is selected from gelling agents and/or inorganic or organic, associative or non associative thickening agents, anionic, non ionic, cationic or amphoteric surfactants, propenetrating agents, emulsifying agents, fragrances, preservatives, fillers, sunscreens, coloring agents, proteins, vitamins, provitamins, moisturizing agents, emollients, softening agents, mineral, vegetal or synthetic oils, hydrophilic or lipophilic active agents such as ceramides and pseudoceramides, antifoaming agents, antiperspirants, free radical scavengers, bactericides, and anti-dandruff agents.

27. Use according to anyone of claims 18 to 26, characterized in that the polymer compound(s) is or are selected from:
polyalkyleneimines, preferably poly((C₂-C₅)alkyleneimines),
polymers that are grafted by a (C₂-C₅)alkyleneimine, preferably polymers that are grafted by the ethyleneimine, more preferably polyamidoamines, crosslinked or not, grafted by the ethyleneimine,
copolymers based on amino (C₁-C₄)alkyl(meth)acrylate, preferably based on aminoethyl(meth)acrylate,
polyallylamines,
polycondensates of at least one compound selected from piperazine, 1-(2-aminoethyl)piperazine, 1,4-bis(3-aminopropyl)piperazine), 1-(C₁-C₂₅)alkyl piperazine, 1,4-di((C₁-C₂₅)alkyl)piperazine, 1-(2-hydroxy((C₂-C₂₅)alkyl))piperazine, imidazole, C₁-C₂₅ alkylimidazole, or combinations thereof, with at least one compound selected from a C₆-C₂₂ alkylene dihalide, an epihalohydrine and/or a C₈-C₂₂ bisepoxide,
polymers containing at least 2 units of one or more basic amino acids, and
dendrimers containing primary amines, preferably polyethyleneimine and polypropyleneimine dendrimers.

28. Use according to claim 27, characterized in that the basic amino acid(s) is or are selected from ornithin, asparagine, glutamine, lysine and arginine.

29. Use according to anyone of claims 18 to 28, characterized in that the polymer compound(s) is or are linear, branched, hyper-branched or dendrimeric.

30. Use according to anyone of claims 18 to 29, characterized in that the hydrophilic segment(s) is or are selected from:
segments of polyhydroxylated compounds, preferably polyalkylene glycol or polyvinyl alcohol segments, and
segments of polycarboxylated compounds.

31. Use according to claim 30, characterized in that polyalkylene glycol segments are selected from polyethylene glycol or polypropylene glycol segments.

32. Use according to anyone of claims 18 to 31, characterized in that the hydrophobic segments are selected from fatty carbon chains.

33. Use according to claim 32, characterized in that fatty carbon chains are selected from C₁₂-C₅₀ fatty alcohols, C₁₂-C₅₀ fatty acids, C₁₂-C₅₀ fatty acid esters.

34. Use according to anyone of claims 18 to 33, characterized in that the hydrophobic and/or hydrophilic segments are grafted onto the polymer compound(s) or sequenced with the amine units.

35. Use according to anyone of preceding claims, characterized in that the modified polymer compound(s) is or are selected from polyethyleneimine-polyethylene glycol, polyethyleneimine-polyvinyl alcohol, polyallylamine-polyethylene glycol, polyallylamine-polyvinyl alcohol, polylysine-polyethylene glycol and polylysine-polyvinyl alcohol.
